(19) United States
(12) Patent Application Publication
(10) Pub. No.: US 2011/0118729 A1
(43) Pub. Date: May 19, 2011

(54) HIGH-INTENSITY PULSED ELECTRIC FIELD VITRECTOMY APPARATUS WITH LOAD DETECTION

(75) Inventors: Tammo Heeren, Aliso Viejo, CA (US); John C. Huculak, Mission Viejo, CA (US); Steven W. Kovalcheck, Aliso Viejo, CA (US)

(73) Assignee: ALCON RESEARCH, LTD, Fort Worth, TX (US)

(21) Appl. No.: 12/618,335

(22) Filed: Nov. 13, 2009

(51) Int. Cl. A61B 18/18

(52) U.S. Cl. 606/41

(57) ABSTRACT

A high-intensity pulsed electric field (HIPEF) vitrectomy apparatus is disclosed. An exemplary apparatus includes a HIPEF probe comprising at least one electrode disposed at a distal end of the HIPEF probe, such that the distal end is configured for insertion into an eye. A load detection circuit is coupled to the HIPEF probe and is configured to compare a measured physical parameter to a corresponding threshold value. A control circuit is electrically coupled to the load detection circuit and configured to selectively disable application of pulsed energy to the at least one electrode of the HIPEF probe, based on the comparison. The measured physical parameter may include, for example, resistivity, permittivity, reflected light, pressure, or heat dissipation capability.

### TECHNICAL FIELD

The present invention relates generally to the field of eye surgery and more particularly to methods and apparatus for performing eye surgery using high-intensity pulsed electric fields.

### BACKGROUND

Techniques and apparatus for dissociation and removal of highly hydrated macroscopic volumes of proteinaceous tissue from the human eye have been previously disclosed. In particular, techniques for dissociation and removal of highly hydrated macroscopic volumes of proteinaceous tissue using rapid variable direction energy field flow fractionation have been disclosed by Steven W. Kovalcheck in “System For Dissociation and Removal of Proteinaceous Tissue”, U.S. patent application Ser. No. 11/608,877, filed 11 Dec. 2006 and published 5 Jul. 2007 as U.S. Patent Application Publ. No. 2007/0156129 (hereinafter “the Kovalcheck application”), the entire contents of which are incorporated herein by reference.

The techniques disclosed in the Kovalcheck application were described in detail in terms of vitreoretinal surgery. However, those of ordinary skill in the art will readily understand that those techniques are applicable to medical procedures in other areas in the body of humans or animals. As explained in the Kovalcheck application, prior art procedures for vitreoretinal posterior surgery have relied for decades on mechanical or traction methods such as: 1) tissue removal with shear cutting probes (utilizing either a reciprocating or rotary cutter); 2) membrane transection using scissors, a blade, or vitreous cutters; 3) membrane peeling with forceps and picks; and 4) membrane separation with forceps and viscous fluids. While improvements in mechanisms, materials, quality, manufacturability, system support, and efficacy have progressed, many of the significant advancements in posterior intraocular surgical outcomes have been primarily attributable to the knowledge, fortitude, skill, and dexterity of the operating ophthalmic physicians.

However, the Kovalcheck application disclosed novel apparatus and methods for delivering a variable direction, pulsed high-intensity and ultra-short duration disruptive electric field (low energy) at a pulse duration, repetition rate, pulse pattern, and pulse train length tuned to the properties of the components of the intraocular extracellular matrix (ECM) to create tissue dissociation. In particular, the Kovalcheck application described a probe for delivering the pulsed rapid disruptive energy field to soft proteinaceous tissue surrounded by the probe. Once the adhesive mechanism between tissue constituents are compromised, fluidic techniques may be used to remove the dissociated tissue.

### SUMMARY

As described more fully below, embodiments of the present invention include a high-intensity pulsed electric field (HIPEF) vitrectomy apparatus that includes a HIPEF probe comprising at least one electrode disposed at a distal end of the HIPEF probe, such that the distal end is configured for insertion into an eye. A load detection circuit is coupled to the HIPEF probe and is configured to compare a measured physical parameter to a corresponding threshold value. A control circuit is electrically coupled to the load detection circuit and configured to selectively disable application of pulsed energy to the at least one electrode of the HIPEF probe, based on the comparison.

In some embodiments, the measured physical parameter is resistivity between first and second electrodes of the HIPEF probe, the load detection circuit is configured to compare measured resistivity to a resistivity threshold value less than an expected resistivity for air but greater than an expected resistivity for vitreous, and the control circuit is configured to disable application of pulsed energy to the first and second electrodes if the measured resistivity is greater than the resistivity threshold value. In some embodiments, the apparatus includes an optical waveguide extending to the distal end of the HIPEF and coupled to an optical sensor in the load detection circuit, and the measured physical parameter is reflected light energy. In these embodiments, the load detection circuit is configured to compare measured reflected light energy to a reflected light threshold value less than an expected reflected light energy for air but greater than an expected reflected light energy for vitreous, and the control circuit is configured to disable application of pulsed energy to the first and second electrodes if the measured reflected light energy is greater than the reflected light threshold value.

In some embodiments, the apparatus includes a pressure sensor coupled to the load detection circuit and configured to measure pressure at or near the distal end of the HIPEF probe, and the measured physical parameter is intraocular pressure. In these embodiments, the load detection circuit is configured to compare measured intraocular pressure to a pressure threshold value greater than an expected pressure value for air but less than an expected pressure value inside the eye, and the control circuit is configured to disable application of pulsed energy to the first and second electrodes if the measured pressure is less than the pressure threshold value. In still other embodiments, the HIPEF vitrectomy apparatus includes a heating element and a temperature sensor disposed at or near the distal end of the HIPEF probe, and the measured physical parameter is temperature. In these embodiments, the load detection circuit is configured to compare a measured temperature to a temperature threshold value less than an expected temperature value for air but greater than an expected temperature value for vitreous, and the control circuit is configured to disable application of pulsed energy to the first and second electrodes if the measured temperature is greater than the temperature threshold value.

In some embodiments, the measured physical parameter is permittivity between first and second electrodes of the HIPEF probe, the load detection circuit is configured to compare measured permittivity to a permittivity threshold value greater than an expected permittivity for air but less than an expected permittivity for vitreous, and the control circuit is configured to disable application of pulsed energy to the first and second electrodes if the measured permittivity is less than the permittivity threshold value.

In some embodiments, the load detection circuit is configured to compare the measured physical parameter to the corresponding threshold value before each application of a burst of pulses to the at least one electrode of the HIPEF probe, and the control circuit is configured to selectively disable the application of each burst of pulses, based on the corresponding comparison. In others, the load detection circuit is instead configured to compare the measured physical parameter to the corresponding threshold value before each application of a single pulse to the at least one electrode of the HIPEF probe, and the control circuit is configured to selectively disable the application of each pulse, based on the corresponding comparison.

Methods for controlling application of high-intensity pulsed electric field (HIPEF) energy during eye surgery are also disclosed. An exemplary method comprises: measuring a physical parameter at or near the distal end of a HIPEF probe, said HIPEF probe comprising at least one electrode disposed at said distal end and configured for delivering pulsed energy to an eye; comparing the measured physical parameter to a corresponding threshold value; and selectively enabling application of pulsed energy to the at least one electrode, based on the comparison. Other methods corresponding to the various load detection circuits summarized above are also disclosed.

Of course, those skilled in the art will appreciate that the present invention is not limited to the above features, advantages, contexts or examples, and will recognize additional features and advantages upon reading the following detailed description and upon viewing the accompanying drawings.

### BRIEF DESCRIPTION OF THE DRAWINGS

FIG. 1 is a perspective view of an exemplary probe used for intraocular posterior surgery.

FIG. 2 is an enlarged perspective view of the tip of the probe shown in FIG. 1.

FIG. 3 is a schematic diagram of high-intensity pulsed electric field (HIPEF) vitrectomy apparatus according to some embodiments of the invention.

FIG. 4 is a schematic diagram of an exemplary load detection circuit according to some embodiments of the invention.

FIG. 5 is a schematic diagram of another exemplary load detection circuit.

FIG. 6 is a schematic diagram of another exemplary load detection circuit.

FIG. 7 is a schematic diagram of still another exemplary load detection circuit.

FIG. 8 is a schematic diagram of yet another exemplary load detection circuit.

FIG. 9 is a process flow diagram illustrating an exemplary method for controlling application of HIPEF energy during eye surgery.

### DETAILED DESCRIPTION

The present disclosure describes an apparatus and method for the dissociation and removal of highly hydrated macroscopic volumes of proteinaceous tissues, such as vitreous and intraocular tissue, during vitreoretinal surgery. More particularly, the techniques disclosed below are directed to methods and apparatus for detecting whether a high-intensity pulsed electric field (HIPEF) probe used for such surgery is actually positioned in an eye, before enabling the application of pulsed energy to the surgical site. Although the techniques disclosed herein are described in detail in terms of instruments and methods for traction-free removal of vitreous and intraocular membranes from the posterior region of the eye without damaging the ultra-fine structure and function of the adjacent or adherent retina, those of ordinary skill in the art will understand the applicability of the disclosed invention for other medical procedures on both humans and animals.

As mentioned above, the Kovalcheck application (U.S. patent application Ser. No. 11/608,877) described a new approach to performing vitreoretinal surgery, using an ultra-short high-intensity directionally changing electrical field rather than classical mechanical means historically used to engage, decompose, and remove vitreous and intraocular tissues. The Kovalcheck application was based on the discovery that a transient change in tissue condition caused by the application of an ultra-short high-intensity directionally changing electrical field is satisfactory for removal of macroscopic volumes of proteinaceous tissue. The technical success of mechanical and liquefying means supports the contention that vitreous material need not be obliterated or disrupted on a molecular level to be removed—rather, an innocuous macroscopic change of state is all that is needed for tissue removal. Accordingly, the removal of intraocular tissue enabled by the techniques described in the Kovalcheck application is traction-free.

The apparatus and method disclosed in the Kovalcheck application cause a local decoupling of the adhesive and structural relations in components of intraocular proteinaceous tissue, through the application of a rapidly changing electrical field. This localized decoupling of the adhesive and structural relations between components of intraocular proteinaceous tissue enables tractionless detachment between intraocular tissue components and the retinal membrane. Fluidic techniques (irrigation and aspiration) may be utilized during the tissue dissociation process to enhance the formation of a high-intensity ultra-short-pulsed electrical field and to remove disrupted tissue at the moment of dissociation. In general, it is intended that only the material within the applied high-intensity ultra-short-pulsed electrical field (also denoted high-intensity pulsed electric field, or HIPEF, herein) is assaulted and removed. Therefore, because only the material assaulted by the applied ultra-short pulses receives the high-intensity ultra-short-pulsed electrical field, there is no far-field effect during the tissue extraction process. This high-intensity ultra-short-pulsed electrical field assault leads to dissociation of the entrained macroscopic volume of intraocular proteinaceous tissue, and then aspiration removes the dissociated entrained macroscopic volume of tissue.

Generally speaking, then, a probe with two or more electrodes is inserted into the target hydrated tissue, vitreous or intraocular tissue. The ends of the electrodes are exposed at the distal end of the probe. An electrical pulse is transmitted down at least one of the electrodes while the other one or more electrodes act as the return conductors. A non-plasma electrical field is created between the electrodes. With each electric pulse, the direction of the created electrical field is changed by reversing polarity of the electric pulse, by electrode switching, or by a combination of both. Pulses may be grouped into bursts, which may be repeated at different frequencies and/or different amplitudes. Such pulse groups may be directed at heterogeneous tissue. The electrical pulse amplitude, duration, duty cycle and repetition rate along with continual changing of field direction, create the disruptive electrical field across the orifice of the aspiration lumen. Tissue is drawn into the orifice of the aspiration lumen by fluidic techniques (aspiration). The tissue is then mixed or diluted with irrigation fluid and disassociated as it traverses the high-intensity ultra-short-pulsed directionally changing electric field. During a given interval, disorder is created in the entrained proteinaceous tissue by changing the direction of the electrical field between one or more of the electrodes at the tip of the probe.

The affected medium between the electrode terminations at the end of the probe consists of a mix of target tissue (e.g. vitreous) and supplemental fluid (irrigation fluid). The electrical impedance of this target medium in which the electrical field is created is maintained by the controlled delivery of supplemental fluid (irrigation fluid). In some embodiments, the supplemental fluid providing the electrical impedance is a conductive saline. The supplemental fluid may be provided by an irrigation source external to the probe, through one or more lumens within the probe or a combination of both. When the supplemental fluid is provided within and constrained to the probe interior, the supplemental fluid may have properties (e.g. pH) and ingredients (e.g. surfactants) that may be conducive to protein dissociation.

The properties of the generated electrical energy field within the target medium are important. In the techniques disclosed in the Kovalcheck application and expanded upon herein, high-intensity, ultra-short pulses (sub-microseconds) of electrical energy are used. Tissue impedance, conductivity and dilution are maintained in the target medium by supplemental fluid irrigation, in some embodiments. The pulse shape, the pulse repetition rate, and the pulse train length may be tuned to the properties of the intraocular tissues, in some embodiments. In some embodiments, multiple pulse patterns may be employed to address the heterogeneity of intraocular tissue.

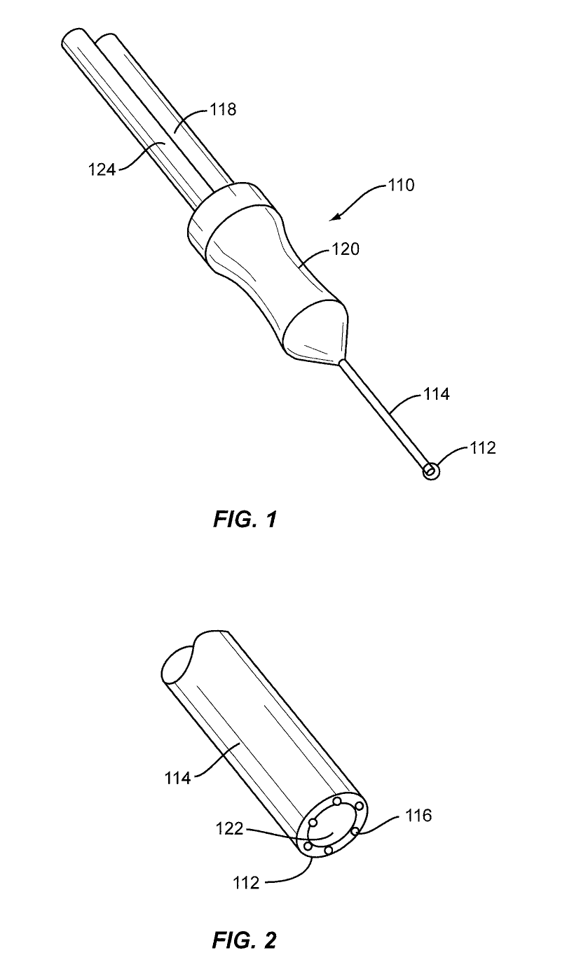

One application of the system described herein is for the treatment of pathologic retinal conditions. An exemplary apparatus for this treatment is shown in FIG. 1, which illustrates a HIPEF probe 110 comprising a hollow probe shaft 114 extending from handle 120 to probe shaft tip 112, an aspiration line 118, and electrical cable/transmission line 124. FIG. 2 illustrates details of the probe shaft 114 and probe shaft tip 112; a plurality of electrodes 116, connected to electrical cable 124, are exposed at the tip 112, and surround an aspiration lumen 122 providing an aspiration pathway to aspiration tube 118.

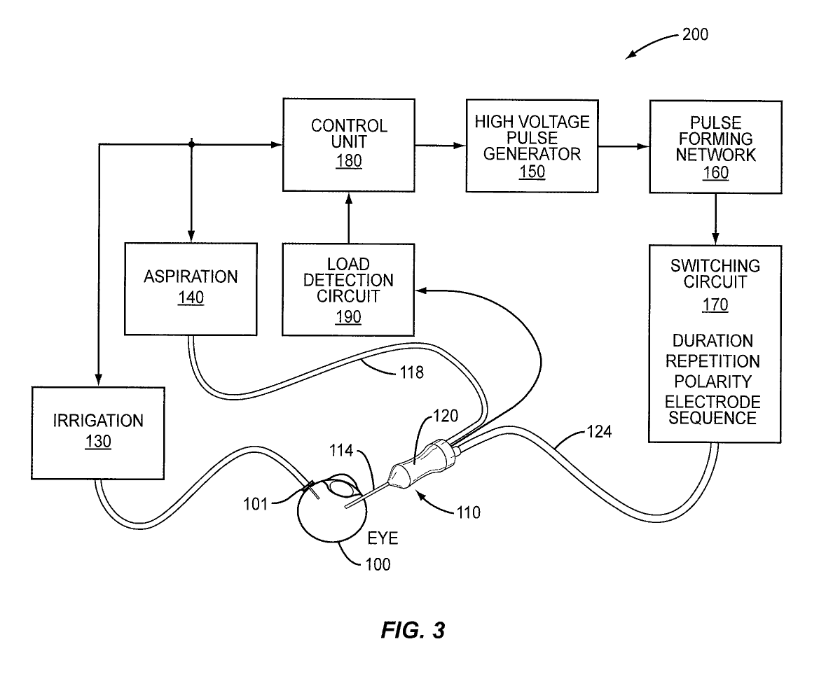

The shaft tip 112 of probe 110 may be inserted by a surgeon into the posterior region of an eye 100 via a pars plana approach 101, as shown in FIG. 3, using handle 120. Using a standard visualization process, vitreous and/or intraocular membranes and tissues are engaged by the shaft tip 112 at the distal end of the hollow probe shaft 114, irrigation 130 and aspiration 140 mechanisms are activated, and ultra-short high-intensity pulsed electric energy from a high voltage pulse generator 150 is delivered through a pulse-forming network 160, switching circuit 170, and cable 124 (which may comprise a transmission line, for example), creating a disruptive high-intensity ultra-short-pulsed electrical field within the entrained volume of tissue. The adhesive mechanisms of the entrained constituents of the tissue that are drawn toward the probe tip 112 via aspiration through an aspiration line 118 connected to an aspiration lumen 122 in the hollow probe shaft 114 are dissociated, and disrupted tissue removed with the aid of the employed fluidic techniques. Engagement may be axial to or lateral to the shaft tip 112 of the hollow probe shaft 114; extracted tissue is removed through the aspiration lumen 122 via a saline aspiration carrier to a collection module.

The apparatus pictured in FIGS. 1 to 3 delivers high-intensity pulsed electric fields (HIPEF) at a pulse duration, repetition rate, pulse pattern, and pulse train length tuned to the properties of the components of the intraocular extracellular matrix. The pulse power generator 150 for the system 200 pictured in FIG. 3 delivers pulsed DC or gated AC against a low impedance presented by the vitreous and the irrigating solution. Included in the system 200 are energy storage, pulse shaping, transmission, and load-matching components. In some embodiments, the peak output voltage of the high voltage generator 150 is sufficient to deliver up to a 300 kV/cm field strength using the electrodes 116 at the distal end 112 of the hollow surgical probe 114 (see FIG. 2). The pulse duration is short relative to the dielectric relaxation time of protein complexes. Further, the pulse duration, repetition rate, and pulse train length (i.e., duty cycle) are chosen to avoid the development of thermal effects (“cold” process). Thus, in some embodiments the system 200 generates and delivers square-shaped or trapezoidal-shaped pulses with rise and fall times of less than five nanoseconds). In some embodiments of the apparatus and method disclosed herein, pulse durations are in the nanosecond range, with voltages produced by pulse forming network 160 greater than one kilovolt and in some cases in the tens of kilovolts.

Switching circuit 170 is configured to control pulse duration and repetition rate, and in some embodiments is configured to generate a stepwise continual change in the direction of the electrical field by switching between electrodes, reversing polarity between electrodes or a combination of both in an array of electrodes at the shaft tip 112 of the hollow probe shaft 114. This continual change in the direction of the electrical field creates disorder in the entrained tissue volume without causing dielectric breakdown of the carrier fluid between the electrodes or thermal effects.

In various applications, the apparatus and techniques described herein may be applied to remove all of the posterior vitreous tissue, or specific detachments of vitreous tissue from the retina or other intraocular tissues or. Engagement, disruption and removal of vitreous tissue, vitreoretinal membranes, and fibrovascular membranes from the posterior cavity of the eye and surfaces of the retina are critical processes pursued by vitreoretinal specialists, in order to surgically treat sight-threatening conditions such as diabetic retinopathy, retinal detachment, proliferative vitreoretinopathy, traction of modalities, penetrating trauma, epi-macular membranes, and other retinopathologies. Though generally intended for posterior intraocular surgery involving the vitreous and retina, it can be appreciated that the techniques described herein are applicable to anterior ophthalmic treatments as well, including traction reduction (partial vitrectomy); micelle adhesion reduction; trabecular meshwork disruption, manipulation, reorganization, and/or stimulation; trabeculoplasty to treat chronic glaucoma; Schlemm's Canal manipulation, removal of residual lens epithelium, and removal of tissue trailers. Applicability of the disclosed apparatus and methods to other medical treatments will become obvious to one skilled in the art, after a thorough review of the present disclosure and the attached figures.

The apparatus of FIG. 3 further includes a load detection circuit 190 electrically connected to probe 110 and control unit 180. (In some embodiments, all or part of load detection 190 may be included in or attached directly to probe 110.) Load detection circuit 190, coupled with the control circuitry in control unit 180, prevents the energizing of the high-intensity pulsed electric fields at the probe shaft tip 112 when the probe shaft tip 112 is not placed in the eye 100. This is important because if the electrodes of an activated probe are exposed to air, rather than vitreous fluid or other intraocular tissue, electrical breakdown may occur. This breakdown may cause damage to the probe and/or the pulse generation circuitry (e.g., switching circuit 170, pulse forming network 160, and high-voltage pulse generator 150). Further, this may cause the unintentional ablation of tissue near the probe tip, and may create free radicals that could have toxic effects.

Generally speaking, the function of load detection circuit 190 is to determine whether or not the one or more electrodes 116 of the probe needle tip 112 are placed in an eye 110, by evaluating a measurement of a physical parameter prevailing at or near the tip. More specifically, load detection circuit 190 is configured in several embodiments of the invention to compare a measured physical parameter such as resistivity, refractivity, pressure, heat dissipation, dielectric constant, or the like, to a corresponding threshold value. Given a pre-determined threshold value suitably situated between the expected measurement value for air and the expected measurement value for vitreous (or other intraocular material), this comparison allows the load detection to determine whether the probe tip is placed in an eye, and to generate a control signal for use by the control circuitry in control unit 180 in selectively disabling the application of pulsed energy to the electrodes.

Load detection can be based on the measurement of one or more of several physical parameters for which the properties of air and vitreous (or balanced salt solution) are sufficiently different to be exploited. These physical parameters include the permittivity (e.g., as expressed by a relative dielectric constant), resistivity, refractive index, and specific heat capacity (e.g., as manifested by a material's ability to dissipate applied heat). Another parameter that may be used is ambient pressure, as the intraocular pressure is normally 15 to 18 mmHg but during Vitreoretinal Surgery could be raised by the physician to 30 to 40 mmHg. Of course, some embodiments may measure two or more of these physical parameters, to enhance the reliability of the load detection.

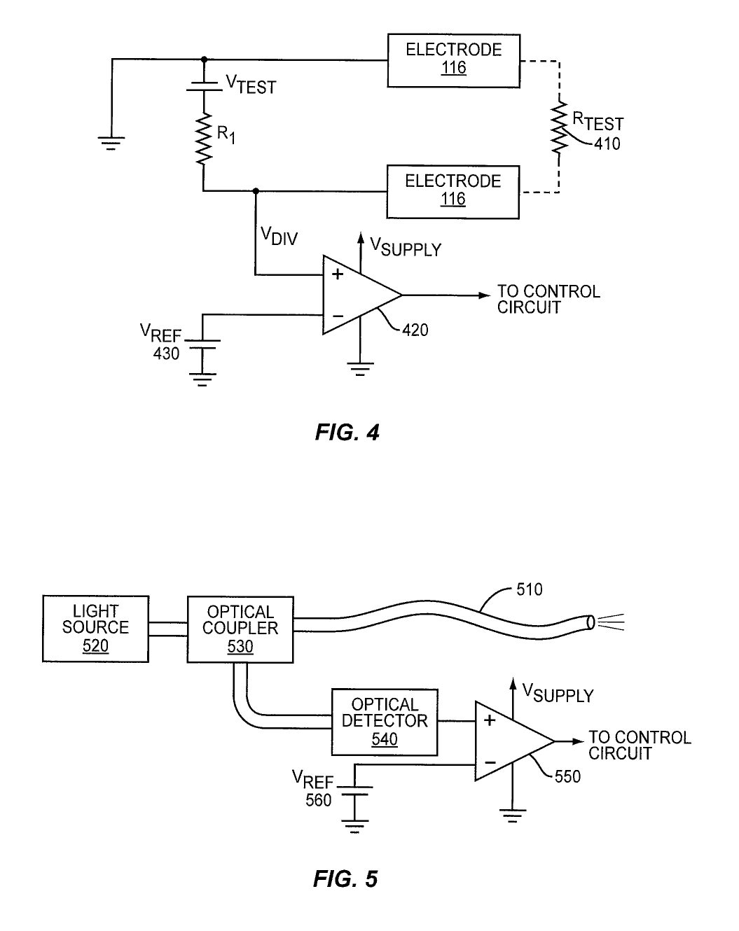

FIG. 4 illustrates an exemplary load detection circuit configured to measure resistivity 410 between two electrodes 116 of a HIPEF probe. The resistivity inherent to air is significantly higher than the resistivity of vitreous or balanced salt solution (BSS). This resistivity can be measured using the same electrodes that are used to apply the pulsed electric field to the eye during surgery. The resistivity is measured before a pulse or burst of pulses, the measured value is compared to a resistivity threshold value less than an expected resistivity for air but greater than an expected resistivity for vitreous or BSS, and the control circuit is selectively disabled if the measured resistivity is less than the resistivity threshold value.

In the circuit of FIG. 4, this is accomplished by applying a test voltage V_{TEST} to the voltage divider network formed by resistor R₁ and the resistivity R_{TEST} between the two electrodes 116. The voltage V_{DIV} presented to the positive terminal of comparator 420 equals R_{TEST}/(R_{TEST}+R₁), and provides a measurement of the resistivity between the electrodes. This voltage is then compared, using comparator 420, to a pre-determined reference voltage V_{REF}, obtained from reference supply 430. A higher voltage V_{DIV} indicates a higher resistivity R_{TEST}. Thus, if V_{DIV}>V_{REF}, the electrodes 116 are likely exposed to air, rather than to vitreous. The resulting “HIGH” output of comparator 420 may be used to disable application of pulsed energy to the electrodes 116. On the other hand, if V_{DIV}<V_{REF}, then the electrodes are likely exposed to vitreous or BSS—the resulting “LOW” output of comparator 420 signals to the control circuit that pulsed energy may be safely applied.

Of course, the circuit in FIG. 4 is only one of many possible circuits for measuring resistivity and comparing the resulting measured value to a pre-determined threshold value. For instance, the circuit in FIG. 4 is entirely analog, with the output of comparator 420 providing a binary control signal (HIGH/LOW) indicating whether or not the measured resistivity exceeds the threshold value. Another circuit could perform the same functions by digitizing V_{DIV}, using an analog-to-digital converter, and comparing the result to a digital threshold resistivity value stored in memory, using a microprocessor, microcontroller, or the like. Still other circuits may employ other mechanisms for sensing the resistivity, such as sensing current flowing from an applied test voltage source to the electrodes, or measuring voltage developed across the electrodes from a known current applied to one of the electrodes. These variations and others will be readily apparent to those of ordinary skill in the art.

Another example of a load detection circuit according to some embodiments of the invention is illustrated in FIG. 5. In this approach, light from light source 520 is generated and supplied to an optical waveguide 510 (e.g., an optical fiber) that extends to the distal end of the probe shaft, i.e., to a point near the probe electrodes. In some embodiments, this waveguide may be inside the probe shaft, while in others it may be attached to the outside of the probe shaft. In any event, a portion of the light that emerges from the end of waveguide 510 is reflected back into the waveguide. The proportion of light that is reflected is a function of the indexes of refraction of the waveguide itself and the material at the end of the waveguide.

The index of refraction of air is about 1.0, whereas the index of refraction of water is about 1.3. It can be assumed that the refractive index of vitreous or BSS is similar to that of water. For a typical fiber optic material, the refractive index is greater than 1.3. Accordingly, if a pulse of light is sent down the waveguide, a larger amount of light will be reflected if air is present at the distal interface than if vitreous or BSS is present. Thus, if the amount of reflected light is below a certain threshold, i.e., a threshold that is less than an expected reflected light energy for air but greater than an expected reflected light energy for vitreous, then a pulse or burst of pulses can be applied.

In the circuit illustrated in FIG. 5, the light reflected back into optical waveguide 510 is separated from the forward-going light by optical coupler 530, and supplied to optical detector 540. The voltage output of optical detector 540 is proportional to the light energy reflected back into optical waveguide 510, and is compared to threshold voltage V_{REF} 560, using comparator 550. V_{REF} 560 is chosen to correspond to a reflected light threshold value that is less than an expected reflected light energy for air, but greater than an expected reflected light energy for vitreous. Thus, comparator 550 provides a “HIGH” output to the control circuit if the output from optical detector 540 is greater than V_{REF} 560, indicating that air is present at the end of the probe and that application of pulsed energy to the probe electrodes should be disabled. If the output from optical detector 540 is less than V_{REF} 560, on the other hand, then the resulting “LOW” output from comparator 550 indicates to the control circuit that the probe electrodes are placed in an eye, and that a pulse or burst of pulses may be applied.

As was the case with the circuit in FIG. 4, those skilled in the art will appreciate that many variations of the circuit of FIG. 5 are possible. For instance, the output of optical detector 540 could be digitized, using an analog-to-digital converter, and compared to an appropriate threshold in the digital domain to generate a control signal for selectively enabling and disabling the application of HIPEF energy to the probe electrodes.

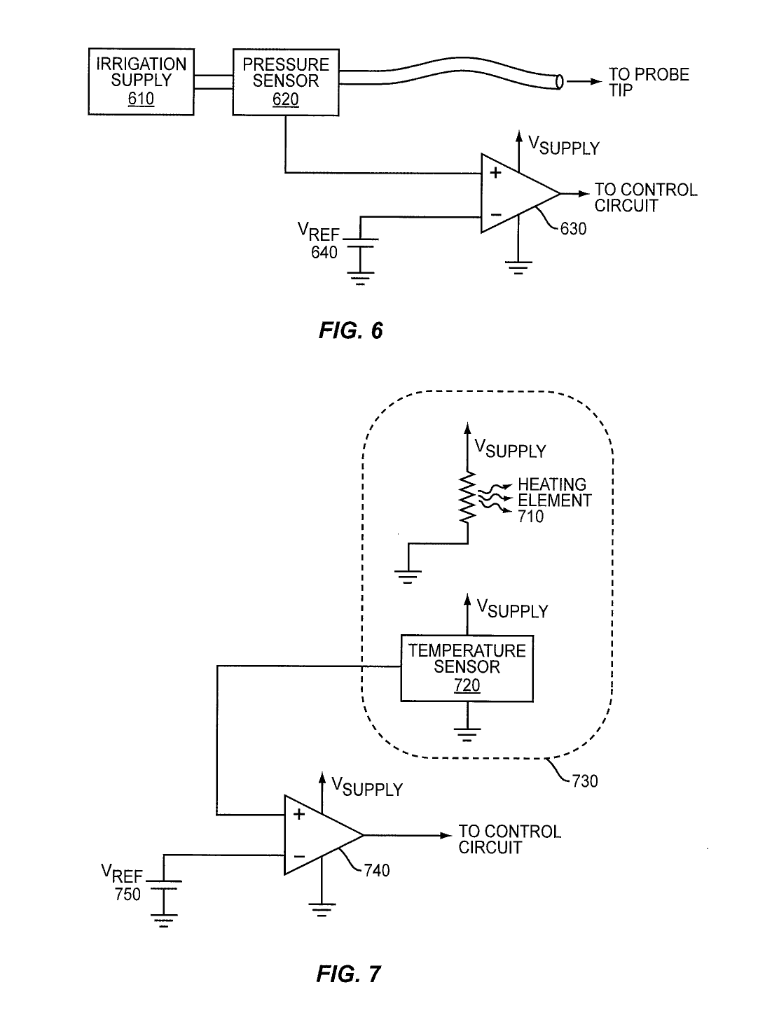

An exemplary circuit based on measuring pressure at or near the end of the probe tip is illustrated in FIG. 6. In the illustrated circuit, a pressure controlled irrigation supply 610 is provided to maintain intraocular pressure to a preset value within the posterior cavity of the eye. The irrigation supply may be separate from the probe (130 in FIG. 3), included in the probe or a combination of both. A pressure sensor 620 which is in communication with the probe tip measures pressure at or near the probe tip. The pressure sensor may incorporate mechanical, fiber optic or piezoelectric properties and may be in communication with the probe tip or be actually located at the probe tip. A measured pressure value from pressure sensor 620 is compared to a pre-determined threshold voltage V_{REF} 640, using comparator 630, and the output passed to the control circuitry to selectively enable or disable application of HIPEF energy to the probe electrodes. In the pictured circuit, assuming that the voltage output of pressure sensor 620 is much lower than the preset irrigation assisted controlled intraocular pressure then a “LOW” output from comparator 630 will indicate that the PEF probe tip may not be in the posterior region of the eye, as the measured pressure will be much lower than the preset intraocular pressure. Of course, many variations of the circuit in FIG. 6 are possible, including variations having opposite output signal polarities.

Still another exemplary load detection circuit is illustrated in FIG. 7. In this case, the load detection function is based on an evaluation of the ability of the medium at the end of the probe to dissipate heat. The heat dissipation capability (e.g., specific heat capacity) of air is significantly lower than that of vitreous or BSS. This difference can be detected using a small heating element 710 (e.g., a resistive heating element) at or near the probe end. By monitoring the temperature rise associated with activating the heating element, using a temperature sensor 720 in close proximity to the heating element 710 (as indicated by dashed line 730), the difference in heat dissipation capability between air and vitreous can be readily detected. The output of temperature 720 is compared to V_{REF} 750, using comparator 740, with the resulting output sent to the control circuit for selectively enabling and disabling the application of HIPEF energy to the probe electrodes.

Of course, those skilled in the art will appreciate that the circuit of FIG. 7 is simplified. In particular, heat dissipation is inherently a delayed process. Thus, the output of temperature sensor 720 should be measured at a particular time relative to the activation of the heating element 710; the necessary timing controls have been omitted from FIG. 7 for simplicity. However, those skilled in the art will readily appreciate the further circuit details needed, and will also appreciate that many variations of this circuit are possible.

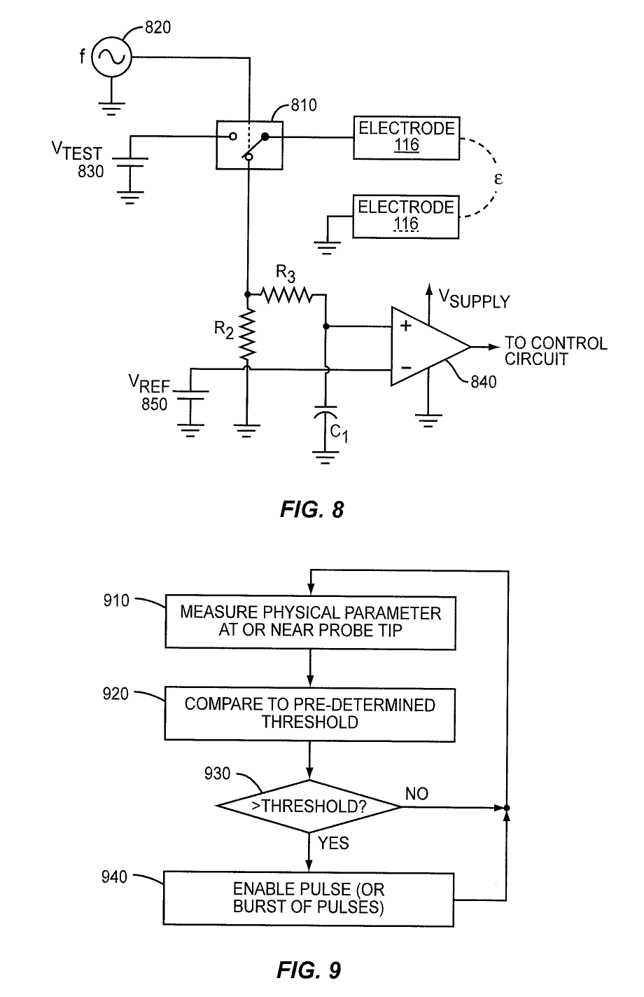

Yet another load detection circuit is illustrated in FIG. 8, this one based on measuring the permittivity of the medium at or near the end of the HIPEF probe. The relative dielectric constant of air is about 1.0, whereas the relative dielectric constant for water is about 80. It can be assumed that the dielectric constant of vitreous or BSS is similar to that of water. As a result, given that the physical configuration of the probe electrodes is constant, a probe tip exposed only to vitreous will observe a higher effective capacitance than a probe tip exposed to air. Thus, the capacitance seen by the probe tip can be measured (whether directly or indirectly), and compared to a pre-determined threshold value to detect whether the probe tip is in air or in vitreous or other fluid.

One circuit for measuring the capacitance seen by the probe tip and comparing the measured result to a threshold value is shown in FIG. 8. Switch 810 is driven by a switching signal from oscillating signal source 820, at a frequency f, so that switch 810 alternately connects one of the probe electrodes 116 to a test voltage V_{TEST} 830 and the positive input of comparator 840. During half of each switching cycle, V_{TEST} 830 is connected to electrode probe 116, and thus “charges” the capacitor produced by the physical configuration of the probe electrodes 116 and the dielectric formed by whichever medium the probe tip is exposed to. During the other half of the switching cycle, the voltage between the probe electrodes 116 is discharged through resistor R₂, at a rate that depends on R₂ as well as the effective capacitance observed by the electrodes 116, which capacitance in turn depends on the permittivity ∈ of the medium observed by the probe tip.

The value of R₂ is chosen, given a switching frequency f, so that the time constant formed by R₂ and the effective capacitance when the probe is in vitreous is less than one-half of the switching period 1/f. Thus, if the probe tip is in air, the effective capacitance is smaller, and the voltage applied to the probe electrode quickly approaches zero during the discharge half of the cycle. On the other hand, if the probe tip is in vitreous, the observed capacitance is larger and the electrode voltage discharges more slowly during the discharge half of the cycle. Thus, the “average” voltage of the probe electrode will be higher when in vitreous. This average voltage is collected by the integrating circuit formed by R₃ and C₁ (which has a time constant substantially longer than a single switching cycle), and compared to threshold voltage V_{REF} 850 with comparator 840. With an appropriately selected V_{REF} 850, the output of comparator 840 is thus “HIGH” if the probe tip is in vitreous, and “LOW” if the probe tip is in air.

Like the circuits discussed earlier, of course, the capacitance-testing load detection circuit of FIG. 8 is but one of several possible circuits that can be used to measure the permittivity of the medium to which the probe electrodes are exposed, and to compare that measurement to a reference threshold to determine whether or not HIPEF energy should be supplied to the probe electrodes. Those skilled in the art will be well aware of alternative circuits, as well as of the performance, cost, and size advantages and disadvantages of each.

Given the various examples of load detection circuits presented above, those skilled in the art will appreciate that FIG. 9 is a process flow diagram illustrating a generalized method for controlling application of high-intensity pulsed electric field (HIPEF) energy during eye surgery, such as might be implemented with any of the circuits described above, variants thereof, or load detection circuits based on measurements based on one or more physical parameters not already discussed. As pictured, the method in FIG. 9 is operated repeatedly, such as before each attempted application of a pulse of HIPEF energy, or before each application of a burst of pulses of HIPEF energy.

Accordingly, each cycle of the pictured method “begins,” as shown at block 910, with the measuring of a physical parameter at or near the distal end of a HIPEF probe, where the HIPEF probe comprises at least one electrode disposed at the distal end and is configured for delivering pulsed energy to an yet. As noted above, the measured physical parameter may be resistivity, refractivity (or reflectivity), pressure, heat dissipation ability (or specific heat capacity), or permittivity. Other physical parameters may be used instead of or in addition to one or more of these, provided only that the measured parameter differs enough between air and vitreous so that an appropriate load detection circuit can distinguish between the two.

As shown at block 920, the measured physical parameter is compared to a pre-determined threshold value, where the threshold value is selected so that it is between the expected measurement value for air and the expected measurement value for vitreous. The exact value of the threshold may be selected to account for expected variations in measurements, including those due to noise from various sources, and/or to provide a desired probability of false detection or false no-detection.

As shown at blocks 930 and 940, if the measured value is greater than the threshold, then the application of a pulse or burst of pulses of energy to the probe electrodes is selectively enabled. On the other hand, if the measured value is less than the threshold value, then application of pulsed energy to the electrodes is not allowed. Instead, the measurement cycle is repeated until the threshold test is met.

Of course, the process illustrated in FIG. 9 assumes that a measured value greater than the threshold value indicates that the probe tip is properly placed in an eye. As seen above in the discussions of FIGS. 4-8, the opposite may be true in some cases. In these cases, of course, the process flow illustrated in FIG. 9 is easily adapted, e.g., by reversing the “YES” and “NO” labels at block 930.

Thus, for example, resistivity may be the physical parameter measured in some embodiments of the process flow of FIG. 9. In this case, application of pulsed energy to the probe electrodes may be selectively enabled only if the measured resistivity is less than a corresponding resistivity threshold value. Similarly, if the measured physical parameter is reflected light from an optical fiber extending to the distal end of the HIPEF probe, then application of pulsed energy to the probe electrodes is selectively enabled only if the measured reflected light is less than a corresponding reflected light threshold value. Likewise, if the measured physical parameter is temperature resulting from generation of heat at or near the distal end of the HIPEF probe, then application of pulsed energy to the probe electrodes is selectively enabled only if the measured temperature is less than a corresponding temperature threshold value, indicating that a material with a greater heat dissipating capability than air is present at the probe tip.

On the other hand, if the measured physical parameter is permittivity (i.e., probe tip capacitance), then application of pulsed energy to the probe electrodes is selectively enabled only if the measured temperature is greater than a corresponding permittivity threshold value is measured. Similarly, if the measured physical parameter is pressure, than energizing the probe electrodes is permitted only if the measured pressure approximately equals the preset intraocular pressure value.

As suggested in the process flow of FIG. 9, the load detection process may be carried out before each application (or attempted application) of pulsed energy to the electrode or electrodes of the HIPEF probe, in which case each pulse is selectively enabled based on a corresponding comparison of the measured physical parameter to the threshold value. Alternatively, the load detection process might be carried out before each application (or attempted application) of a burst of pulses, in which case each burst may be selectively enabled based on the corresponding comparison of the measured parameter to the threshold value.

Those skilled in the art will appreciate that the control signal generated by the process flow of FIG. 9 and/or the load detection circuits of any of FIGS. 4-8 may be used to selectively enable and/or disable the application of pulse energy to the HIPEF probe electrodes in any of a number of different ways. Referring once more to FIG. 3, for example, a control circuit in control unit 180 may respond to the control signal supplied by load detection circuit 190 by completely disabling high-voltage pulse generator 150, such as by shutting down or opening a switch to a power supply, for example. Of course, those skilled in the art will appreciate that other techniques for selectively disabling and/or enabling the application of pulsed energy to HIPEF probe 110 are possible; further illustration of these possibilities is not necessary to a complete understanding of the present inventive techniques.

Indeed, all of the preceding descriptions of various methods and apparatus for controlling the application of high-intensity pulsed electric field energy during eye surgery were given for purposes of illustration and example, and those skilled in the art will appreciate that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A high-intensity pulsed electric field (HIPEF) vitrectomy apparatus, comprising:
a HIPEF probe comprising at least one electrode disposed at a distal end of the HIPEF probe, wherein the distal end is configured for insertion into an eye;
a load detection circuit coupled to the HIPEF probe and configured to compare a measured physical parameter to a corresponding threshold value; and
a control circuit electrically coupled to the load detection circuit and configured to selectively disable application of pulsed energy to the at least one electrode, based on the comparison.

2. The HIPEF vitrectomy apparatus of claim 1, wherein the measured physical parameter is resistivity between first and second electrodes of the HIPEF probe, the load detection circuit is configured to compare measured resistivity to a resistivity threshold value less than an expected resistivity for air but greater than an expected resistivity for vitreous, and the control circuit is configured to disable application of pulsed energy to the first and second electrodes if the measured resistivity is greater than the resistivity threshold value.

3. The HIPEF vitrectomy apparatus of claim 1, further comprising an optical waveguide extending to the distal end of the HIPEF and coupled to an optical sensor in the load detection circuit, and wherein the measured physical parameter is reflected light energy, the load detection circuit is configured to compare measured reflected light energy to a reflected light threshold value less than an expected reflected light energy for air but greater than an expected reflected light energy for vitreous, and the control circuit is configured to disable application of pulsed energy to the first and second electrodes if the measured reflected light energy is greater than the reflected light threshold value.

4. The HIPEF vitrectomy apparatus of claim 1, further comprising a pressure sensor coupled to the load detection circuit and configured to measure pressure at or near the distal end of the HIPEF probe, and wherein the measured physical parameter is intraocular pressure, the load detection circuit is configured to compare measured intraocular pressure to a pressure threshold value greater than an expected pressure value for air but less than an expected pressure value for vitreous, and the control circuit is configured to disable application of pulsed energy to the first and second electrodes if the measured pressure is less than the pressure threshold value.

5. The HIPEF vitrectomy apparatus of claim 1, further comprising a heating element and a temperature sensor disposed at or near the distal end of the HIPEF probe, and wherein the measured physical parameter is temperature, the load detection circuit is configured to compare a measured temperature to a temperature threshold value less than an expected temperature value for air but greater than an expected temperature value for vitreous, and the control circuit is configured to disable application of pulsed energy to the first and second electrodes if the measured temperature is greater than the temperature threshold value.

6. The HIPEF vitrectomy apparatus of claim 1, wherein the measured physical parameter is permittivity between first and second electrodes of the HIPEF probe, the load detection circuit is configured to compare measured permittivity to a permittivity threshold value greater than an expected permittivity for air but less than an expected permittivity for vitreous, and the control circuit is configured to disable application of pulsed energy to the first and second electrodes if the measured permittivity is less than the permittivity threshold value.

7. The HIPEF vitrectomy apparatus of claim 1, wherein the load detection circuit is configured to compare the measured physical parameter to the corresponding threshold value before each application of a burst of pulses to the at least one electrode of the HIPEF probe, and wherein the control circuit is configured to selectively disable the application of each burst of pulses, based on the corresponding comparison.

8. The HIPEF vitrectomy apparatus of claim 1, wherein the load detection circuit is configured to compare the measured physical parameter to the corresponding threshold value before each application of a single pulse to the at least one electrode of the HIPEF probe, and wherein the control circuit is configured to selectively disable the application of each pulse, based on the corresponding comparison.

9. A method for controlling application of high-intensity pulsed electric field (HIPEF) energy during eye surgery, the method comprising:
measuring a physical parameter at or near the distal end of a HIPEF probe, said HIPEF probe comprising at least one electrode disposed at said distal end and configured for delivering pulsed energy to an eye;
comparing the measured physical parameter to a corresponding threshold value; and
selectively enabling application of pulsed energy to the at least one electrode, based on the comparison.

10. The method of claim 9, wherein measuring the physical parameter comprises measuring resistivity between first and second electrodes of the HIPEF probe, and wherein selectively enabling application of pulsed energy comprises enabling application of pulsed energy to the first and second electrodes if the measured resistivity is less than a corresponding resistivity threshold value.

11. The method of claim 9, wherein measuring the physical parameter comprises measuring reflected light from an optical waveguide extending to the distal end of the HIPEF probe, and wherein selectively enabling application of pulsed energy comprises enabling application of pulsed energy to the at least one electrode if the measured reflected light is less than a corresponding reflected light threshold value.

12. The method of claim 9, wherein measuring the physical parameter comprises measuring pressure at or near the distal end of the HIPEF probe, and wherein selectively enabling application of pulsed energy comprises enabling application of pulsed energy to the at least one electrode if the measured pressure is greater than a corresponding pressure threshold value.

13. The method of claim 9, further comprising generating heat at or near the distal end of the HIPEF probe, and wherein measuring the physical parameter comprises measuring temperature at or near the distal end of the HIPEF probe and selectively enabling application of pulsed energy comprises enabling application of pulsed energy to the at least one electrode if the measured temperature is less than a corresponding temperature threshold value.

14. The method of claim 9, wherein measuring the physical parameter comprises measuring permittivity between first and second electrodes of the HIPEF probe, and wherein selectively enabling application of pulsed energy comprises enabling application of pulsed energy to the first and second electrodes if the measured permittivity is greater than a corresponding permittivity threshold value.

15. The method of claim 9, wherein the physical parameter is measured before each application of two or more bursts of pulses to the at least one electrode of the HIPEF probe, and wherein each burst is selectively enabled based on a corresponding comparison of the measured physical parameter to the threshold value.

16. The method of claim 9, wherein the physical parameter is measured before each application of a single pulse to the at least one electrode of the HIPEF probe, and wherein each pulse is selectively enabled based on a corresponding comparison of the measured physical parameter to the threshold value.
